(19) United States
(12) Patent Application Publication
(10) Pub. No.: US 2024/0016411 A1
(43) Pub. Date: Jan. 18, 2024

(54) PERSONALIZED BREATH TRAINING SYSTEM

(71) Applicant: Halare, Inc., Sewickley, PA (US)

(72) Inventors: Anthony C. Warren, Wien (AT); Kyle H. Goldschmidt, Saint Louis Park, MN (US)

(21) Appl. No.: 18/031,203

(22) PCT Filed: Oct. 14, 2021
(86) PCT No.: PCT/US2021/054910

§ 371 Date: Apr. 11, 2023

Related U.S. Application Data
(60) Provisional application No. 63/092,049, filed on Oct. 15, 2020.

(51) Int. Cl. A61B 5/08; A61B 5/00; G16H 20/30

(52) U.S. Cl. CPC A61B 5/0816; A61B 5/6898; A61B 5/7264; A61B 5/486; G16H 20/30; A61B 2505/09

(57) ABSTRACT

A portable smart device designed to be held by a user or worn by the user for determining a breathing quality of a user during a breath training session. The portable smart device including a sensor configured to detect a breathing parameter of the user and output a breathing parameter signal. The breathing parameter including at least one of heart rate, heart rate variability, breathing rate, breathing rate variability, vagal tone, breath holding capability, variations in breath holding capability, blood oxygen saturation level and blood oxygen saturation level variability. The portable smart device also including and a processor configured to analyze the breathing parameter signal of the user during the breath training session to determine breathing parameter values, set the determined breathing parameter values as a baseline for the user, and output a semi-personalized breath training routine to the user based on baseline.

### TECHNICAL FIELD

The present disclosure relates to systems and methods for personalized breath training.

### BACKGROUND

There is significant interest to improve the general health and quality of life of the population by enhancing healthcare delivery systems in terms of quality, outcomes and costs. Also, there is a growing awareness by individuals that, by improving their lifestyles and wellness, they may prolong their lives, mitigate the symptoms of existing chronic disease states, delay the onset of chronic disease states and lower the long-term impact of such diseases and their treatment while reducing overall healthcare costs.

There is considerable clinical evidence that improving breathing behavior by undertaking breathing training exercises can entirely alleviate or at least reduce the symptoms of several chronic health problems including, but not limited to asthma, snoring, sleep apnea, COPD, PTSD, panic attacks, anxiety and stress, hyperventilation syndrome, speech disorders, and withdrawal from addictive agents.

Breathing exercises may also improve general health and wellness states as well as athletic performance.

### SUMMARY

A portable smart device designed to be held by a user or worn by the user for determining a breathing quality of a user during a breath training session. The portable smart device including a sensor configured to detect a breathing parameter of the user and output a breathing parameter signal. The breathing parameter including at least one of heart rate, heart rate variability, breathing rate, breathing rate variability, vagal tone, breath holding capability, variations in breath holding capability, blood oxygen saturation level and blood oxygen saturation level variability. The portable smart device also including and an electronic computing unit coupled to the sensor. The electronic computing unit including a processor configured to analyze the breathing parameter signal of the user during the breath training session to determine breathing parameter values, set the determined breathing parameter values as a baseline for the user, and output a semi-personalized breath training routine to the user based on baseline.

A method for determining a breathing quality of a user during a breath training session, where a portable smart device is held by a user or worn by the user. The method includes detecting, by a sensor, a breathing parameter of the user and outputting a breathing parameter signal. The breathing parameter includes at least one of heart rate, heart rate variability, breathing rate, breathing rate variability, vagal tone, breath holding capability, variations in breath holding capability, blood oxygen saturation level and blood oxygen saturation level variability. Analyzing, by a processor of an electronic computing unit coupled to the sensor, the breathing parameter signal of the user during the breath training session to determine breathing parameter values, setting, by the processor, the determined breathing parameter values as a baseline for the user, and outputting, by the processor, a semi-personalized breath training routine to the user based on baseline.

### BRIEF DESCRIPTION OF THE DRAWINGS

The disclosure is best understood from the following detailed description when read in connection with the accompanying drawings, with like elements having the same reference numerals. When a plurality of similar elements is present, a single reference numeral may be assigned to the plurality of similar elements with a capital letter designation referring to specific elements. Included in the drawings are the following figures:

FIG. 1 is a block diagram of a system for performing and assessing breath training sessions in TRAINEES, according to aspects of the disclosure.

FIG. 2A shows an example device for performing and assessing breath training sessions in TRAINEES, according to aspects of the disclosure.

FIG. 2B shows an example device for performing and assessing breath training sessions in TRAINEES, according to aspects of the disclosure.

FIG. 2C shows an example device for performing and assessing breath training sessions in TRAINEES, according to aspects of the disclosure.

FIG. 2D shows an example device for performing and assessing breath training sessions in TRAINEES, according to aspects of the disclosure.

FIG. 3 is an example table showing the description of exercise tiers, according to aspects of the disclosure.

FIG. 4 is a flowchart showing steps of assessing, classifying and personalization of breath training for a TRAINEE, according to aspects of the disclosure.

FIG. 5 is a portion of an example classification table, according to aspects of the disclosure.

FIG. 6 is another portion of an example classification table, according to aspects of the disclosure.

### DETAILED DESCRIPTION

To maximize the benefit from breathing training exercises, the breathing exercises should be personalized to match the needs of the individual TRAINEE (i.e., user of the breath training system) taking into account a number of factors which include but are not limited to: a) their existing breathing behavior, b) the symptoms that they wish to reduce or alleviate, or the health status or athletic level they wish to achieve, and c) certain physiological and personal factors, referred to throughout this application as “PERSONAL PROFILE” including but not limited to, height, weight, age, gender, ethnicity, location, daily activity level, sleep patterns, use of additive substances, allergies and other health related factors and history thereof

Currently, there are insufficient qualified breath trainers to provide personalized training to a large population which could benefit from such training. Additionally, the cost of an individual having a personalized breath trainer is high and may be economically out of reach from TRAINEES most likely to benefit from the training. Furthermore, travel to a breath training clinic may not be possible for the majority of TRAINEES from reasons of accessibility, cost or both. For breath training to be effective it is important that a TRAINEE complies with the personalized breath training instructions when not under the personal guidance of a trainer.

The methods/systems described herein meet the goals described above inclusive specifically with regard to: a) determining the current breathing behavior parameters of a TRAINEE before commencing training, such parameters being referred to as BASELINE throughout this application, b) recording a PERSONAL PROFILE of said TRAINEE so that the TRAINEE can be categorized as one of a large number of pre-determined CLASSIFICATIONS, c) creating a personalized breath training exercises to meet the defined training goals based on the CLASSIFICATION of the TRAINEE, their BASELINE, the benefits and targets from the training and their progress during training, d) encouraging the TRAINEE to comply closely to the personalized breath training course of exercises proscribed, and e) providing all these features in an economically viable way.

There is no existing system by which TRAINEES can be trained in breathing behavior with personally designed breath training exercises, guided in their training with modifications made to the training based on progress, and be incentivized to continue with the training in a location of their choice and outside a professional environment.

The present disclosure relates to systems and methods for aiding TRAINEES wishing to improve their breathing behavior by undertaking personalized breath training exercises at a place of their choice without the need for a specialized trainer. Training exercises are created to match specific needs of an individual TRAINEE based on an analysis of: a) BASELINE breathing behavior parameters of the TRAINEE, b) data stored in a data repository from a multiplicity of other TRAINEES, and/or from published data on breathing behavior and training benefits, c) progress of the TRAINEE during training, d) TRAINEE compliance to training instructions during training or, e) from a combination of two or more from a), b), c), and d). Data records on TRAINEE identity, compliance to personalized exercises and TRAINEES' progress are aggregated in a central data repository while preferably retaining the confidentiality of TRAINEES. The data are analyzed to provide feedback to TRAINEES from time to time.

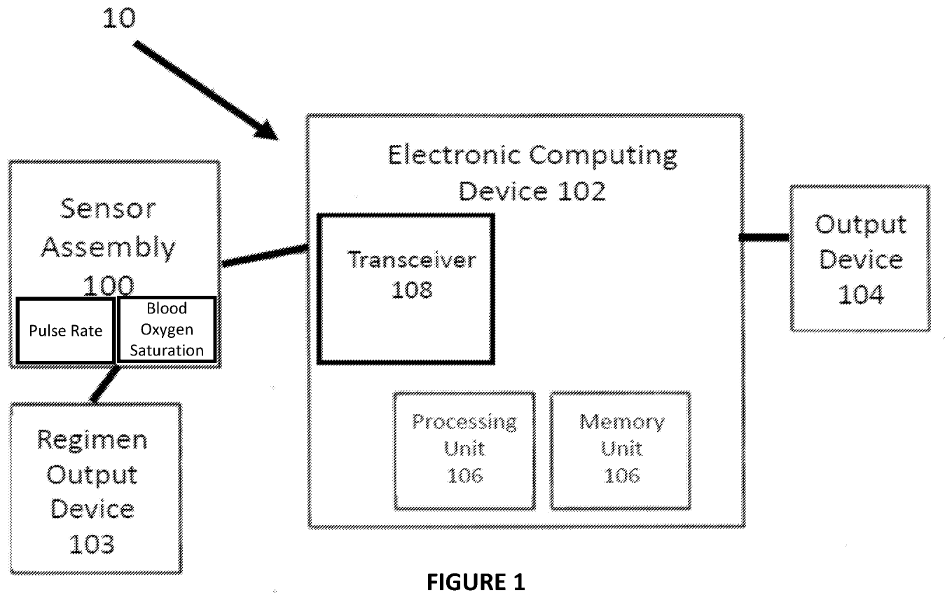

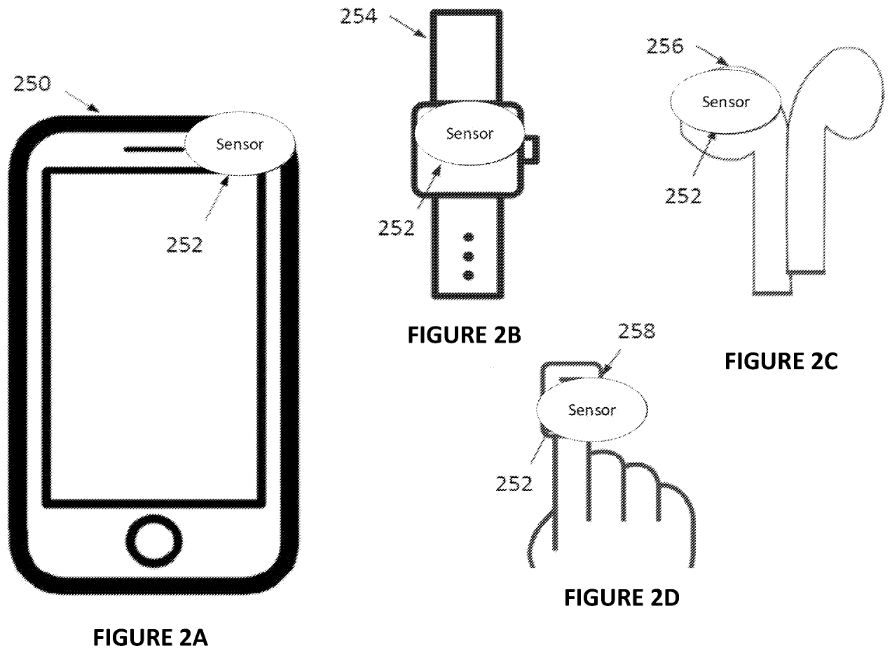

Referring to FIG. 1, a block diagram of a system 10 is shown for capturing the physiological parameters of the TRAINEE, analyzing the physiological parameters. The physiological parameters include but not be limited to: heart rate, heart rate variability, breathing rate, breathing rate variability, vagal tone, breath holding capability, variations in breath holding capability, blood oxygen saturation level and variability. Generally, the system 10 includes a sensor assembly 100, an electronic computing device 102, and an output device 104 (e.g., display screen, speaker, etc.). Although the sensor assembly 100, the electronic computing device 102, and output device 104 are depicted as separate components in system 10, it is contemplated that any or all of these components may be integrated together in two or one device. For example, the sensor assembly, the electronic computing device 102, and the output device 104 may be integrated into an apparatus attachable to a TRAINEE (e.g., a wristband, a neckband, other attachments, etc.), or in a smart device, such as a smart phone, tablet computer, laptop computer, etc. Examples of such devices are shown in FIG. 2A as smartphone 250 with a camera and light source acting as a sensor 252 (e.g., heart rate sensor and/or pulse oximeter), FIG. 2B as smartwatch 254 with camera and light source or electrodes acting as a sensor 252 (e.g., heart rate sensor and/or pulse oximeter), FIG. 2C as earbuds 256 with camera and light source or electrodes acting as a sensor 252 (e.g., heart rate sensor and/or pulse oximeter) and FIG. 2D as a finger clamped device 258 (e.g., heart rate sensor and/or pulse oximeter). Other suitable sensors for detecting physiological data of a TRAINEE will be understood by one of ordinary skill in the art from the description herein.

System 10 includes a regimen output device 103. The regimen output device 103 is adapted to output instructions to the TRAINEE according to the breath training sessions established for the TRAINEE to detect physiological data of the TRAINEE. The regimen output device 103 may be coupled to the sensor assembly 100, or may be a separate component, such as a smart phone, tablet computer, laptop computer, or other communication device capable of providing breath training instructions to the TRAINEE.

The system 10 further includes an electronic computing device 102 with a processing unit 106 (e.g., including a processor), a transceiver 108, and a memory unit 110. The transceiver 108 may be utilized to receive physiological data detected from the sensor assembly 100. In embodiments where the electronic computing device 102 is integrated with the sensor assembly 100, the transceiver 108 may not be a necessary component for the transmission and reception of data to be analyzed by the electronic computing device 102. The memory unit 110 is depicted as integrated into the electronic computing device 102. It is contemplated that additional memory units may be utilized, such as a memory unit integrated into the sensor assembly 100 or a cloud storage device. Such memory units are configured to store detected physiological data and subsequent analyzed data.

The processing unit 106 is adapted to process the data detected by the sensor assembly 100 according to particular algorithms to classify the TRAINEE into a breathing category and guide the TRAINEE to improve their classification. The particular algorithms the processing unit 106 applies to the physiological data detected by the sensor assembly 100 depend upon the type of data detected and the sensors that are used to detect the data. Although the algorithms described herein are related to an individual sensor type, the physiological data analyzed from each type of sensor may be used in conjunction with or in combination with data from other sensors to determine breathing quality.

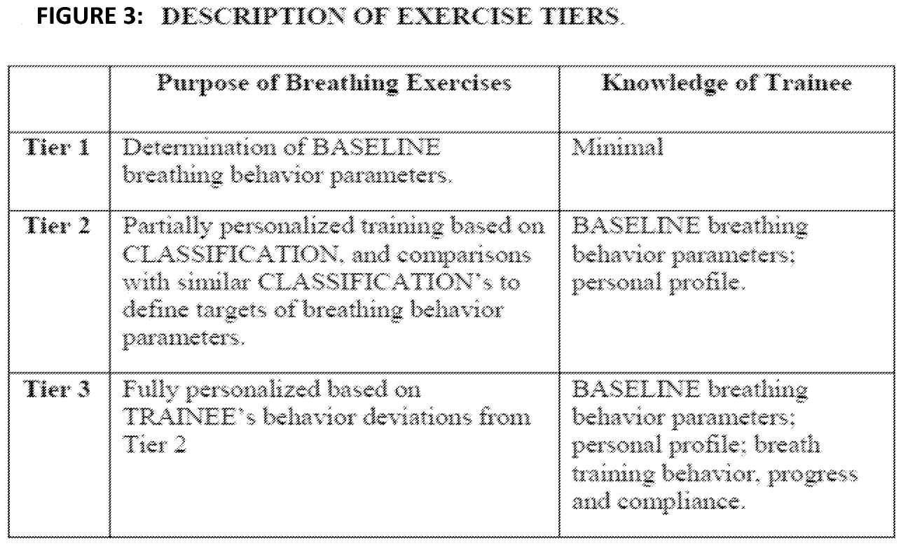

As more information is acquired about the TRAINEE, the exercises can be increasingly personalized. For the purpose of clarity, it is convenient to define exercises into three tiers, as the personalization is increased. FIG. 3 describes these tiers and their purpose. As shown in Tier 1, the system determines the TRAINEE BASELINE breathing behavior parameters. At Tier 2, the system determines a partially personalized training routine for the TRAINEE based on classification of the TRAINEE based on their BASELINE breathing behavior parameters. At Tier 3, the system determines a fully personalized training routine for the TRAINEE based on deviations in breathing behavior parameters as compared to the classification in Tier 2 (e.g., how the TRAINEE begins to deviate from the known parameters of the particular class).

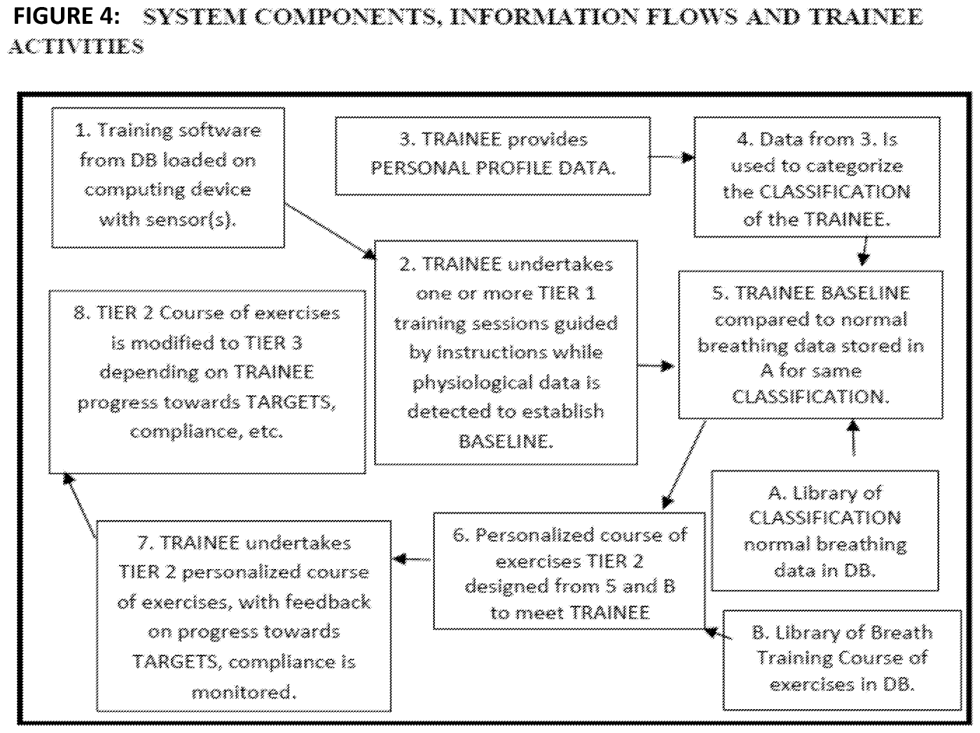

FIG. 4 is an example of the steps taken to establish and perform personalized breath training exercises:

Step 1: A central data repository or database (DB), stores software programs designed to provide personalized breath training. Programs are downloaded and installed in a portable computing device from time to time. The device either has one or more sensors capable of detecting certain physiological data, or has the means to communicate in real time to such sensors, or can operate in both modes.

Step 2: Before creating a personalized course of exercises for a new TRAINEE, the system determines the breathing behavior parameters of the TRAINEE. This is achieved by a TIER 1 program which guides the TRAINEE through breathing exercises, where the TRAINEE is instructed to control their breath in a certain manner (e.g., holding their breath for a duration, breath in a specific pattern, etc.), during which the sensor or sensors detect certain physiological data which are then stored in the portable device or uploaded to the database in a central server which may work in conjunction with the smart device to perform the processing steps described in FIG. 4. This initial set of breathing behavior parameters determined by the TIER 1 program is referred to as the BASELINE parameters. The TRAINEE may undertake a single or several sessions guided by the TIER 1 program. The greater the number of sessions, the higher the accuracy of the BASELINE data. The physiological data used to determine the TRAINEEs BASELINE breathing behavior parameters are processed either by the portable computing device or by a central computing apparatus. Parameters determined by the analysis may include but not be limited to: heart rate, heart rate variability, breathing rate, breathing rate variability, vagal tone, breath holding capability, variations in breath holding capability, blood oxygen saturation level and variability. The TRAINEE may also answer one or more questionnaires designed to determine levels of daytime drowsiness such as the Epworth Sleepiness Scale and similar scales. Such questionnaires help in determining whether sleep disturbed breathing may be affecting daytime alertness. Other questionnaires such as the Asthma Control Questionnaire (ACQ) can be used for other applications.

Step 3: The TRAINEE also provides personal data which may include but is not limited to: age, gender, ethnicity, weight, height, body mass index, level of daily physical activity, level and frequency of snoring events, level and frequency of sleep apnea and asthma events, level of allergies including allergic rhinitis, level of smoking, chronic diseases, and health history. These data in aggregate are referred to as PERSONAL PROFILE

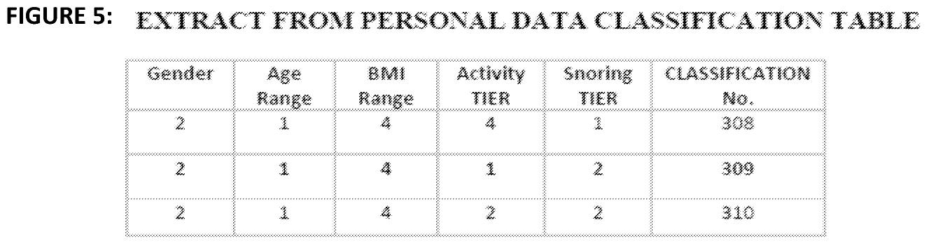

Step 4: The PERSONAL PROFILE is used to categorize the TRAINEE into a unique CLASSIFICATION. Based on the number of personal data fields listed above, there may be many (e.g., several thousand) categories of CLASSIFICATION. FIG. 5 shows an example extracted from a CLASSIFICATION look-up table with, in this example, a reduced set of personal data and the resultant CLASSIFICATION for the TRAINEE. In this example the TRAINEE has been asked to specify the following personal attributes; gender, age in one of 10 age ranges, height and weight, which are used to calculate Body Mass Index (BMI) then assigned to one of five ranges, their daily level of physical activity from a choice of five levels, and how often they snore from a choice of five ranges. In this example the classification table stored in a central database has a total of 2,500 CLASSIFCATIONs, of which 308, 309 and 310 are shown in FIG. 5. In this method each TRAINEE is matched to a single CLASSIFICATION based on the personal data provided (e.g., the personal data provided is compared to the data in the classification table to determine the best match). The single CLASSIFICATION is then used to determine the appropriate breath training course for the TRAINEE. If additional personal data fields are used, the number of CLASSIFICATIONs will increase.

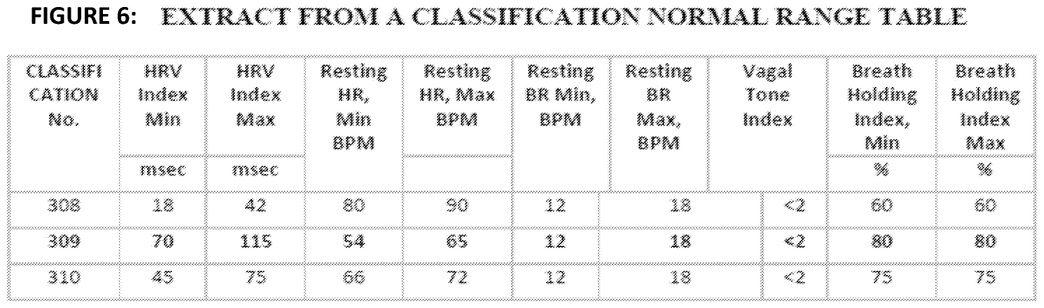

Step 5: The central DB also has a data table (A) of CLASSIFICATIONs, each unique CLASSIFICATION matched with breathing behavior parameters for normal healthy individuals. These values are referred to herein as “norms” and “norm parameters”. Norms for healthy individuals for different CLASSIFICATIONs are published in the research literature. Typically these parameters will have a range. For example a TRAINEE categorized as CLASSIFICATION 390 may be expected to have a base heartrate in the range 54 to 65 beats per minute, and a relaxed breathing rate in the range 12-18 breaths per minute. The BASELINE breathing behavior parameters of the TRAINEE categorized say as CLASSIFICATION 390 are compared with the standard CLASSIFICATION range of values for the same category 390. FIG. 5 shows extracted portions from a table showing CLASSIFICATION categories derived from certain personal data and FIG. 6 shows an extracted portion from a table listing CLASSIFICATION norms of breathing related data for the same selection of CLASSIFICATIONs. The difference between the TRAINEE' s measured breathing parameters and the norm parameters is used to create a set of TARGET values for the TRAINEE to acquire when performing breath training exercises.

Step 6: Based on the comparison described above, breath training exercises designed for the specific TRAINEE is derived from the stored data table (B) of exercises stored in the central DB. Breath training exercises are based on independent data reported from research studies. For example,. there are multiple peer reviewed studies that report the most appropriate breathing exercises for the alleviation of asthma, for the alleviation of sleep disturbed breathing, for managing anxiety and for training for specific sports. Exercises may include different components in varied sequences, For example, for the alleviation of sleep disturbed breathing a sequence including 2-3 minutes of relaxed slow breathing interspersed with breath holding, and suppression of hyperventilation is repeated 5 or 6 times over a period of twenty minutes. For the relief of anxiety and the control of panic attacks, exercises designed to reduce the rest breathing rate to 6 breaths a minute by introducing small breath holds after each exhale are used. TRAINEES are also guided in how to breath correctly with emphases on nasal and diaphragm usage. Exercises may be undertaken sitting upright in a quiet environment, or may be taken during exercise. Combinations and derivations from the stored training exercises may be created. This TIER 2 exercise is not fully personalized at this stage, as the system still needs to monitor the behavior of the TRAINEE undertaking the TIER 2 exercises. As an example of TIER 2 exercises, a TRAINEE CLASSIFICATION that has sleep disturbed breathing symptoms reported in their personal data, and has low breathing holding capability compared with the CLASSIFICATION norm, and a low vagal tone compared with the same CLASSIFICATION norm, would be proscribed, for example, a 12 week training course of exercises of exercises, including both breath holding and relaxed breathing sequences interspersed with each other. The targets set would be within the norms from the stored CLASSIFICATION norm table.

Likewise, a TRAINEE wishing to cease the use of nicotine and showing higher heartrate, breathing rate, and vagal tone than their CLASSIFICATION norms, together with a breath holding capability lower than their CLASSIFICATION norm may have a semi-personalized course of exercises for exercises including two stages. The first stage would focus on increasing breath holding control to a value known to be necessary prior to a cessation attempt. The second stage, is started after the targets in stage one are reached, would focus on relaxed slow breathing to help with anxiety attacks during withdrawal.

Step 7: The TRAINEE is guided through training sessions defined by the TIER 2 Course of exercises of exercises while physiological data, as listed in step 2 above, are detected, stored and analyzed. The TRAINEE can access their session data together with improvement trends towards their personal targets derived from the CLASSIFICATION database. The ability to see how their training is improving their breathing behavior parameters against healthy comparable CLASSIFICATIONS incentivizes TRAINEES to comply with the training exercises and instructions therein. The system also monitors and stores one or more behaviors of the TRAINEE. These may include but are not limited to: a) the completion of any session, b) date and time of day of session, c) failure to undertake a session, or undertaking too many sessions within a recommended period, d) incorrect performance of breathing exercises, e) improvements in one or more breathing parameters towards targets exceeding expectations, and f) improvements in one or more breathing parameters towards targets not meeting expectations.

Step 8: The TIER 2 course of exercises of exercises is modified as appropriate to a TIER 3, fully personalized breath training course of exercises of exercises by taking into account the monitored and stored behaviors from Step 7. Modifications in Step 8 may continue throughout this step as the behavior of the TRAINEE is continually monitored. As an example, if the TRAINEE's progress is exceeding expectations, the TRAINEE may be instructed to miss a day in every 5 days of training, or instructed to terminate the exercises early, or to move the targets to more challenging values, or to increase or decrease the exercises' length, or to add other breath training exercises. If the TRAINEE is experiencing difficulties and not achieving progress as expected towards one or more TARGET, the exercises may be adjusted to help the TRAINEE overcome difficulties by, for example, modifying the course of exercises to alleviate the difficulties, reduce the targets, increase the number and/or the length of sessions. Other means to continually personalize the exercises based on the TRAINEE's behavior, progress and TARGETS will be understood to somebody versed in the art and, as such, are included as part of this application.

The steps defined above may also apply to TRAINEES undertaking meditation or mindfulness sessions in which breathing is used as part or all of the training methods. The steps taken by a TRAINEE are provided here as an illustrative example.

A TRAINEE may be wishing to improve one or more of the following health issues: a) increase athletic ability, b) reduce or eliminate the symptoms of rhinitis, asthma, COPD, sleep apnea, anxiety, panic attacks, hyperventilation syndrome, c) cease using additive substances such as nicotine and heroin d) other breathing related issues, and e) meditation of mindfulness activities, has access to a device which is equipped with one or more physiological sensors. For this illustration a smart-phone (see FIG. 2A) equipped with a camera is used. Other so-called wearable devices equipped with suitable sensors may serve the same purpose, or again sensor modules that can communicate with a portable computing device. Such sensors may include finger clips, ear clips, watches, ear-buds and others.

The TRAINEE places a body part against the sensor(s) in the device. In this embodiment the trainee places a finger on the camera of the smart-phone. If the sensors are integral with a so-called smart watch, the body part would be the wrist. A software program, specifically designed to analyze a TRAINEE'S existing breathing behavior is stored in the central computer (e.g., server) and is either downloaded to the sensor equipped device or the device has real-time access to the program over a data network. This program is designated herein as a TIER 1 course of exercises.

The purpose of the TIER 1 course of exercises is to determine the current breathing behavior of the TRAINEE, recorded as a set of breathing behavior parameters and referred to herein as BASELINE. The TRAINEE is guided with prompts to undertake certain breathing instructions while keeping a finger on the camera. Such instructions may include breath holding, relaxed breathing, slow breathing, variation in breathing rate, variation in breathing depth, inhale/exhale ratio changes, and others. Typically a sequence of instructions may continue for up to ten minutes and longer. The sensor detects one or more physiological data from the camera such as the heart beat signature (e.g., PPG data) and blood oxygen saturation level. The trainee may be instructed to remove and/or replace their finger at different times during the exercises. It may be beneficial to undertake several exercise sessions like this in order to acquire sufficient data for an accurate analysis.

During or after the completion of the breathing exercises, the data collected from the sensor, in this case the camera, is analyzed to extract a number of breathing behavior parameters including some or all of the following and possibly others not listed here and referred to in total as the BASELINE:
a) Resting heart rate and heart rate variability,
b) Amplitude and periodicity of the PPG signal,
c) Resting breathing rate and changes in breathing rate,
d) Breath holding capability after exhale, after normal inhale and after a deep inhale,
e) Changes in blood oxygen saturation levels,
f) Vagal tone ratio, an indicator of the balance of arousal between the sympathetic and parasympathetic branches of the central nervous system.

The TRAINEE may also be asked to provide further data that may be related to breathing behavior. These include but are not limited to:
a) Impact of sleep deprivation on daytime drowsiness possibly derived using a questionnaire.
b) Pattern of asthma attacks and their severity using a questionnaire.
c) Physical factors such as weight, height, gender, age, and ethnicity.
d) Amount of regular physical activity.
e) Smoking habits and previous attempts to cease usage.
f) Level and frequency of snoring episodes.
g) Level and frequency of sleep apnea episodes.
h) Level and frequency of nasal allergies.
i) Other health related issues and history.

The data described above in part or in their entirety are used to determine a CLASSIFICATION category for the TRAINEE. There may be several thousand independent CLASSIFICATION categories depending on the number of variables and the number of values for each variable.

The TRAINEE's BASELINE is compared to the stored data of normal values for healthy persons for such data for the same or similar CLASSIFICATION using a multi-variable look-up table. The comparison determines the differences between the TRAINEE's breathing behavior parameters with those of a healthy or fit person with the same CLASSIFICATION. For example, the TRAINEE may have symptoms of irregular breathing and a high daytime drowsiness index compared with a healthy person with the same CLASSIFICATION.

Based on the comparison of the TRAINEE's data and that of healthy individuals, the system creates a semi-personalized breath training course of exercises for the TRAINEE from stored exercises. Modifications and derivatives of the stored exercises may be utilized. Such a course of exercises is referred to as TIER 2. As one example, if a TRAINEE has poor breath holding capacity and reports a high level of cigarette use, and wishes to cease nicotine use, a two-stage course of exercises will be created. The first stage will be aimed at extending the breath holding capability to a target level which has been shown to be needed by that CLASSIFICATION category to increase the chance of cessation prior to attempting cessation. Once reaching that level, the TRAINEE will be taken to the second course of exercises to improve their vagal tone which will help in combating withdrawal anxiety. Such a two-stage course of exercises has the potential of doubling the chance of successful withdrawal from nicotine with or without an adjunct method such as nicotine patches.

As the TRAINEE follows the TIER 2 course of exercises, they may not comply within an exercise session or not perform exercise sessions. Additionally, they may not be progressing towards the TARGETS as expected, or are exceeding the TARGETS as expected. The system monitors behaviors and changes in one or more breathing behavior parameters and can adjust the course of exercises and instructions thereby personalizing these fully to maximize the benefits to the TRAINEE. Exercises that are fully personalized to a TRAINEE are referred to herein as TIER 3. As one example, if a TRAINEE is not extending their breath holding time to meet the TARGET range for this parameter, the course of exercises can be modified to concentrate entirely on this problem until the TARGET is reached. As a second example, if a TRAINEE has not improved their vagal tone sufficiently, the course of exercises can be modified to concentrate entirely on relaxed breathing by using a paced breathing addition to the instructions. Or again as a third example, if a TRAINEE is exceeding one or more TARGETS, they can be rewarded by either having rest days from the course of exercises, or shortening the course of exercises, or both.

The system communicates with the TRAINEE, indicating how their progress is compared with others like them based on the stored data from others in their CLASSIFICATION or CLASSIFICATIONS that have significant commonalities. Such peer comparisons are known to promote compliance.

1. A portable smart device designed to be held by a user or worn by the user, the portable smart device for determining a breathing quality of a user during a breath training session, the portable smart device comprising:
a sensor configured to detect a breathing parameter of the user and output a breathing parameter signal, the breathing parameter including at least one of heart rate, heart rate variability, breathing rate, breathing rate variability, vagal tone, breath holding capability, variations in breath holding capability, blood oxygen saturation level and blood oxygen saturation level variability; and
an electronic computing unit coupled to the sensor, the electronic computing unit comprising a processor configured to:
analyze the breathing parameter signal of the user during the breath training session to determine breathing parameter values,
set the determined breathing parameter values as a baseline for the user, and output a semi-personalized breath training routine to the user based on baseline.

2. The portable smart device of claim 1, wherein the processor is further configured to:
compare the breathing parameter values to stored breathing parameter values to classify the user into a classification; and
output a personalized breath training routine to the user based on the classification.

3. The portable smart device of claim 2, wherein the processor is further configured to classify the user into the classification based on personal information of the user, the personal information including at least one of age, gender, height, weight, daily activity, snoring levels and medical history.

4. The portable smart device of claim 2, wherein the stored breathing parameter values include normal value ranges for other users in the classification.

5. The portable smart device of claim 2, wherein the processor is further configured to output the personalized breath training routine as a course of breath training exercises with targets based on the classification.

6. The portable smart device of claim 2, wherein the processor is further configured to reclassify the user into a new classification when during a subsequent breath training session, new breathing parameter values of the user deviate from the breathing parameter values used for the classification.

7. The portable smart device of claim 5, wherein the processor is further configured to modify the personalized breath training routine with a modified course of breath training exercises and modified targets in response to user progress.

8. The portable smart device of claim 5, wherein the breath training exercises include both breath holding sequences and relaxed breathing sequences interspersed with each other.

9. A method for determining a breathing quality of a user during a breath training session, where a portable smart device is held by a user or worn by the user, the method including:
detecting, by a sensor, a breathing parameter of the user and outputting a breathing parameter signal, the breathing parameter including at least one of heart rate, heart rate variability, breathing rate, breathing rate variability, vagal tone, breath holding capability, variations in breath holding capability, blood oxygen saturation level and blood oxygen saturation level variability; and
analyzing, by a processor of an electronic computing unit coupled to the sensor, the breathing parameter signal of the user during the breath training session to determine breathing parameter values,
setting, by the processor, the determined breathing parameter values as a baseline for the user, and
outputting, by the processor, a semi-personalized breath training routine to the user based on baseline.

10. The method of claim 9, further comprising:
comparing, by the processor, the breathing parameter values to stored breathing parameter values to classify the user into a classification; and
outputting, by the processor, a personalized breath training routine to the user based on the classification.

11. The method of claim 10, further comprising:
classifying, by the processor, the user into the classification based on personal information of the user, the personal information including at least one of age, gender, height, weight, daily activity, snoring levels and medical history.

12. The method of claim 10, wherein the stored breathing parameter values include normal value ranges for other users in the classification.

13. The method of claim 10, further comprising:
outputting, by the processor, the personalized breath training routine as a course of breath training exercises with targets based on the classification.

14. The method of claim 10, further comprising:
reclassifying, by the processor, the user into a new classification when during a subsequent breath training session, new breathing parameter values of the user deviate from the breathing parameter values used for the classification.

15. The method of claim 13, further comprising:
modifying, by the processor, the personalized breath training routine with a modified course of breath training exercises and modified targets in response to user progress.

16. The method of claim 13, wherein the breath training exercises include both breath holding sequences and relaxed breathing sequences interspersed with each other.
